(19) United States
(12) Patent Application Publication
(10) Pub. No.: US 2021/0283019 A1
(43) Pub. Date: Sep. 16, 2021

(54) Reporting Pill Dispenser

(71) Applicant: Glenn Whitfield Nelson, Dallas, TX (US)

(72) Inventor: Glenn Whitfield Nelson, Dallas, TX (US)

(21) Appl. No.: 16/974,346

(22) Filed: Oct. 3, 2019

(51) Int. Cl. A61J 7/00; A61J 1/03; A61B 5/024; A61B 5/145; A61B 5/021; A61B 5/0205; G06F 21/32

(52) U.S. Cl. CPC A61J 7/0084; A61J 1/03; A61B 5/024; G06F 21/32; A61B 5/021; A61B 5/02055; A61J 7/0069; A61B 5/14542

(57) ABSTRACT

Reporting Pill Dispenser for tracking, reminding, and assuring pills regimen execution, includes: container holding plurality of pills prescribed, cap permanently affixed to container, an assembly for the pill regimen management system, and a pill dispensing mechanism. Pill management system, can dispense one pill or one dosage at a time, includes: Pill Dispensing Mechanism controlled by a Microcontroller; a reminder system to inform the User to take pills; and accounting system to keep track of pills in the container, dispensed, and not dispensed; a Bio-Sensor to measure the heart rate, oxygen levels, blood pressure and temperature of the User; a Fingerprint Reader to authenticate the User; all contained in the cap of the Pill Container. The pill management system communicates with a User Database where all the pill regimen information is retained. This User Database provides information to the Physicians, Pharmacy, and Family members the status of the User's pill regimen.

### BACKGROUND OF THE INVENTION

It is common for users to not take their pills as prescribed by their Physician, Pharmacy, or Hospitals, or to overdose pill prescribed, these admissions are grouped in under dosage or over dosage characteristics.

Prescription under dosage, poll shows that almost 50% of people forget to take their medication at least once a month. User adherence to medication regimes is a large problem across the World. Across all medicines, it has been estimated that up to 75% of people do not take their medicines properly.

When asked, users often give the following reasons for forgetting to take their medication altogether or at the wrong time in the past month:

Over 20% had forgotten to take their pills once in the last month.

Over 25% had forgotten twice or more in the last month to take their pills.

Over 15% had taken their pills at the wrong time once in the last month.

Over a quarter said they had taken their pills at the wrong time twice or more in the last month.

Older adults, people with disabilities, and caregivers may encounter challenges when taking pill prescriptions. Resolving these problems can lead to much better results from pill medications.

Caregivers often alert their Physician and Pharmacists of the user difficulties in taking their medications, including the following:

Difficulty remembering to take medications.

Difficulty reading labels on prescription labels and over-the-counter products.

Difficulty in scheduling many different medications throughout the day. One of the greatest challenges for older persons and caregivers is working medication schedules into daily routines.

Three out of four adults acknowledge not always taking their medicines as directed. Poor adherence to medicines takes many forms, and nearly 75% of adults report engaging in one or more non-adherent behaviors. While the most common form of non-adherence is simply forgetting to take a prescribed medicine, almost 30% of users stop taking their medicine before it runs out and another third of users report not taking a prescription to the pharmacy to be filled or refilled. Among users who fill their prescriptions, 70%-75% do not take their medicines as prescribed, meaning they skip doses, take less than the recommended amount, or stop taking the medicine earlier than they are instructed to do so. Chronic disease affects nearly one in two Americans and treating chronically ill users' accounts for 75% out of every dollar spent on medical care in America. Treatment often involves following medication regimens over long periods of time to slow disease progression and prevent costly complications. Monitoring studies indicate that among chronically ill users who fill their prescriptions, only about half actually take their medicines as directed by their Physicians.

Prescription Overdose, according to the Centers for Disease Control (2019 CDC), More than 70,200 Americans died from drug overdoses in 2017, including illicit drugs and prescription opioids—a 2-fold increase in a decade. Drug overdose deaths rose from 16,849 in 1999 to 70,237 in 2017. The sharpest increase occurred among deaths related to fentanyl and fentanyl analogs (other synthetic narcotics) with more than 28,400 overdose deaths.

These facts show that this is a huge problem in America and the World, there is a need for a device that helps reduce the amount of under dose from prescription drugs and prevent overdose deaths. Product available are costly, too difficult to use, and generally require a manual programming of the prescription regimen.

Therefore, it would be advantageous to provide a personalized, Reporting Pill Dispenser that reminded the user when to take the prescription and one that informed others (Physician, Pharmacy, or Hospitals 135 and Family members 134) when they did not take their prescription. This Reporting Pill Dispenser should also report when prescriptions are running out and it's time to renew, therefore the pharmacy would be notified, and the renewed prescription could be mailed or come by the pharmacy to refill.

This invention can help to relieve this pill taking regimen, by alerting the user when to take the medicine, report to Physician, Pharmacy, or Hospitals 135 and Family members 134 when the regimen is not maintained, and report to Physician, Pharmacy, or Hospitals 135 when the prescription is in need or renewal.

### FIELD OF THE INVENTION

This Reporting Pill Dispenser is designed for tracking the dispensing of medications related to the controlled dispensing of prescription drugs and over-the-counter medications to users. The system provides the capability to detect successful execution of scheduled and unscheduled pill dispensing events, and to report those events to a User Database which summarizes and reports the events to the prescribing Physician, Pharmacy or Hospital and Family members. These actions are to manage the scheduled pill dispensing and report to interested parties when the pill dispensing regimen is not followed for the medications within the Pill Container.

### BRIEF SUMMARY OF THE INVENTION

A Reporting Pill Dispenser device for dispensing pills, capsules, or tablets “pills” includes: a container of a multitude of sides to hold the plurality of pills prescribed by the Physician, Pharmacy, or Hospitals 135 and; a cap when applied is permanently affixed to the container (not removable), the electronics for the pill dispenser are included in the cap along with the Pill Dispensing Mechanism 108. The pill dispenser can dispense one pill at a time or one dosage at a time, includes; a pill dispensing mechanism controlled by a Microcontroller 125; a reminder system to inform the user when pills are to be taken; an accounting system to keep track of pills dispensed and not dispensed; a Bio-Sensor 109 device to measure the general health of the user; a Fingerprint Reader 119 to verify the authenticity of the user; all contained in the cap of the Reporting Pill Dispenser.

Components of the Reporting Pill Dispenser include a Microcontroller 125 which keeps track of the pill regimen; a Pill Dispenser Mechanism 108 to serve one pill at a time or one dosage at a time; a Real Time Clock 140 for alerts and reminder timings; a counter to count the pills in the container and pills dispensed; an Information Display 101 to interface with the User 128; a Bio-Sensor 109 to measure heart rate, calculate oxygen levels, calculate blood pressure, and measure the thumb temperature with the Skin Temperature Sensor 117; a Fingerprint Reader 119 to verify user authenticity by the User Authentication Device 100; a Tilt Sensor 120 to know the relative position of the pill dispenser; visual indicators to inform the User 128 of status; an Audible Alert Device 138 to alert the User 128; a WiFi wireless interface 133 to connect to the User Database 136; and an internal Communication Bus 139 to interconnect the Reporting Pill Dispenser components. The Reporting Pill Dispenser also include; a method to manage the pill regimen and keep track of the pill dispensed and not dispensed; a transceiver for Bluetooth wireless interface 132 to connect to a Smartphone 127; a WiFi wireless interface 133 to connect to the Internet 141 for communication to the User Database 136; and multiple of input switches to interface to the Microcontroller 125 of the Reporting Pill Dispenser.

The pill dispenser enables user compliance to the pill prescription regimen by providing alerts and reminders when to take the pills; and a method to communicate to the User Database 136 of user metrics and status; to keep track of pills taken and pills not taken, and alert other parties of the users' Family members 134, or Physician, Pharmacists, or Hospitals 135 when the user dispenses pills or not dispenses pills, or are in need of a prescription refill. When the pill dispenser is inverted (dumping, upside down) or angled downward, pills will fall into the pill sorting funnel, one pill or one dosage amount is dispensed by the Pill Dispenser Mechanism 108 at a time. Once dispensed the internal system tallies the pills dispensed to keep track of the pills regimen, reporting to the User Database 136 on an event base, hourly, daily or weekly routine. The pill dispenser can report pill taking attempts and events that are outside the prescription regimen time period indicating user anxiety for medication, these pill taking attempts or events may lead to additional health diagnoses of the health for the User.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that though some figures of the accompanying drawings are drawn to scale, other embodiments may vary in scales and measurements of the various elements, and the emphasis is instead placed on illustrating the principles of the invention. The invention will now be described, by way of example, with reference to the accompanying drawings in which:

FIG. 1 illustrates an isometric view of the Reporting Pill Dispenser, according to a first embodiment of the present invention;

FIG. 2 illustrates a view of the multitude of different pill container sizes for the Reporting Pill Dispenser, the Pill Dispenser Cap 104 remains consistent for all pill container sizes, to hold a plurality of pills prescribed, Pill Container 110 is designed to hold 30 pills within the container, Pill Container 111 is designed to hold 60 pills within the container, Pill Container 112 is designed to hold 90 pills within the container (henceforth, the Pill Container 110, 111, and 112 will be referred to as Pill Container 110 where all features and descriptions apply to all the Pill Containers);

FIG. 3 illustrates a top view of the Reporting Pill Dispenser of FIG. 1;

FIG. 4A illustrates a bottom view of the Reporting Pill Dispenser showing the Pill Funnel 113 of FIG. 1, FIG. 4B shows a side view of the Pill Dispenser Cap 104 showing the Pill Funnel 113 in relation to the other components of the Pill Dispenser Cap 104 (side view);

FIG. 5 illustrates the Cap Retention Mechanism 114 of the Pill Dispenser Cap 104 and the Pill Container 110 of FIG. 1;

FIG. 6A illustrates the Bio-Sensor 109 of FIG. 1, showing the Bio-Sensor Touch Sensor 115 of the Bio-Sensor 109 in FIG. 6A; showing the Bio-Sensor 109 without the Bio-Sensor Touch Sensor 115 in FIG. 6B;

FIG. 7 illustrates the User Authentication Device 100 of FIG. 1, showing the User Authentication Touch Sensor 118 and Fingerprint Reader 119 of the User Authentication Device 100;

FIG. 8 illustrates a bottom view of the Reporting Pill Dispenser of FIG. 1, without the Pill Funnel 113 shown. The bottom side of the Microcontroller Assembly 126 consists of two Batteries 121 to power the Reporting Pill Dispenser, the Tilt Sensor 120, The Pill Dispenser Mechanism 108, the Motor 124 and Pulley 122, the Pill Slot 105, the Pill Slot Sensor 123, the Bio-Sensor 109 and its associated components Skin Temperature monitor 117 and Heart Rate Monitor 116, the User Authentication Device 100;

FIG. 9 illustrates a top view of the Microcontroller Assembly 126 of the Reporting Pill Dispenser of FIG. 1, without the Pill Dispenser Cap 104 top cover shown. The top side of the Microcontroller Assembly 126 consists of the Microcontroller 125, the Tilt Sensor 120, The Pill Dispenser Mechanism 108, the Pill Slot 105, the Position Sensor 130, the Bio-Sensor 109 and its associated components Skin Temperature monitor 117 and Heart Rate Monitor 116, the User Authentication Device 100, the Information Display 101, the YES Switch 102 and No Switch 106;

FIG. 10 illustrates the user inverting (dumping) the Reporting Pill Dispenser and the Pill Dispenser Mechanism 108 spinning the Motor 124 under control of the Microcontroller 125 signaled by the Tilt Sensor 120 to expel a pill from the interior of the Pill Container 110 to the exterior of the Reporting Pill Dispenser;

FIG. 11A illustrates a view of the Reporting Pill Dispenser without the Pill Dispenser Cap 104. In the FIG. 11A view, the Bio-Sensor 109, Heart Rate Monitor 116, and Skin Temperature monitor 117 can be seen in plain view. FIG. 11B identified in the circle of FIG. 11A is a closer view of the Bio-Sensor 109 components. FIG. 11B illustrates a close up view of the Bio-Sensor 109 identifying the Bio-Sensor Switch 129, Heart Rate Monitor 116, and Skin Temperature monitor 117 of FIG. 11A;

FIG. 12 illustrates an isometric view of the Pill Dispenser Mechanism 108;

FIG. 13 illustrates an isometric view of the Pill Tray 137 for the Pill Dispenser Mechanism 108;

FIG. 14 illustrates an isometric view of the Pill Slot 105 for the Pill Dispenser Mechanism 108;

FIG. 15 illustrates a close up view of the Pill Dispenser Mechanism 108 show in the Pill Dispenser Cap 104, Motor 124 and Pulley 122 driving the Pill Tray 137 of FIG. 13. The Microcontroller Assembly 126 consists of the Microcontroller 125, the Pill Dispenser Mechanism 108, the Bio-Sensor 109, the User Authentication Device 100, the Information Display 101, and the Pill Container 110;

FIG. 16 illustrates the user holding the Reporting Pill Dispenser between his thumb and index finger of the left hand (the right hand can also be used to hold the Reporting Pill Dispenser), to view the Information Display 101 of the Reporting Pill Dispenser. This illustrated view of the Reporting Pill Dispenser is the normal view the user has of the Reporting Pill Dispenser;

FIG. 17 showing the block diagram of the Microcontroller Assembly 126, the Microcontroller 125, the Tilt Sensor 120, the Pill Dispenser Mechanism 108, the Motor 124, the Position Sensor 130, and the Pill Slot Sensor 123, the Bio-Sensor 109 and its associated components, Skin Temperature Sensor 117, and Heart Rate Monitor 116, and the Bio-Sensor Touch Sensor 115, the User Authentication Device 100 and its associated Fingerprint Reader 119, and User Authorization Touch Sensor 118, the Information Display 101, the YES Switch 102 and No Switch 106, and Bio-Sensor Switch 129, and the Audible Alert Device 138, all the components of the Microcontroller Assembly 126 communicate with the Microcontroller 125 by way of the Communications Bus 139 and various control pins of the Microcontroller 125, the Real Time Clock 140 is contained within the Microcontroller 125;

FIG. 18 shows the overall communications systems for the Reporting Pill Dispenser. The Reporting Pill Dispenser communicates by the Bluetooth wireless interface 132 to a Smartphone 127 for pill regimen status, uploads to the Reporting Pill Dispenser configuration, and alert monitoring. The Reporting Pill Dispenser communicates by the WiFi wireless interface 133 to the Home Router 131 which communicates to the Internet 141 which communicates with the User Database 136, where prescriptions are downloaded to the Reporting Pill Dispenser from the User Database 136 and status, uploads, and alerts monitoring from the Reporting Pill Dispenser are uploaded to the User Database 136. The User Database 136 is the central location for all data related to the Reporting Pill Dispenser. The User Database 136 communicates to the Internet 141 which communicates to the Family members 134 for status, downloads from the User Database 136, and alert monitoring from the User Database 136. The User Database 136 communicates to the Internet 141 which communicates to the Physician, Pharmacy, or Hospitals 135 for status, downloads from the User Database 136, and alert monitoring for the Physician, Pharmacy, or Hospitals 135 from the User Database 136. Prescription upload from the Physician, Pharmacy, or Hospitals 135 is received in the User Database 136 which is downloaded to the Reporting Pill Dispenser to begin the execution of the Pill regimen;

FIG. 19 illustrates the user holding the Reporting Pill Dispenser in his right hand. The Reporting Pill Dispenser can be handled in either the right hand or the left hand. The Reporting Pill Dispenser is used with the Information Display 101 at the top, with the Yes Switch 102 and No Switch 106 accessible by the other hand;

FIG. 20A and FIG. 20B illustrates the general usage flow between the Reporting Pill Dispenser and the user, this flow chart that illustrates how the Microcontroller Assembly 126 of one embodiment of the presently claimed Reporting Pill Dispenser will send information to the User Database 136 and/or receive instructions from the User Database 136;

### DETAILED DESCRIPTION OF THE INVENTION

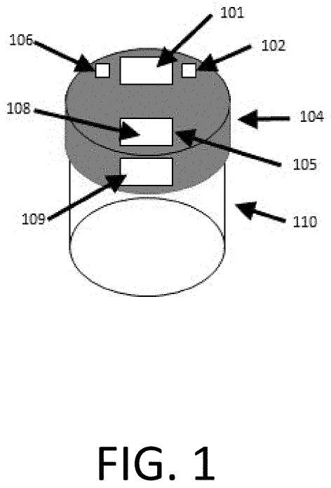

FIG. 1 illustrates an isometric view of the Reporting Pill Dispenser equipped with the Pill Dispenser Cap 104 that is attached to the Pill Dispenser Container 110. The Pill Dispenser Cap 104 is further comprised with the Information Display 101, a Yes Switch 102, a No Switch 106, the Pill Dispensing Mechanism 108, the Pill Slot 105, and the Bio-Sensor 109. The User Authentication Device 100 is not visible in FIG. 1.

The Reporting Pill Dispenser includes a Pill Dispenser Container 110 where the pills are housed, and a Pill Dispenser Cap 104 which is initially detached, once applied to the container the cap becomes affixed to the container, non-removable. The Pharmacy 135 dispensing the medication would fill the Pill Dispenser Container 110 per the prescription, then apply the Pill Dispenser Cap 104.

The Reporting Pill Dispenser once the User 128 is verified, administers a pre-prescribed pill dosage amount at a pre-prescribed time, one pill at a time or one dosage at a time. The Pill Dispenser Cap 104 contains the Microcontroller Assembly 126 which verifies the User's 128 authentication by the User Authentication Device 100, reminds User 128 when pills are to be taken, and informs the Family members 134, Physician, Pharmacy, or Hospitals 135 when pills are dispensed, when pills are not dispensed, and notifies when the prescription is in need of renewal.

The Reporting Pill Dispenser is activated (comes alive) when pills are to be dispensed, a WiFi wireless interface 133 provides connectivity to the User Database 136 which relays pill dispenser status information to User Database 136, a counter within the Microcontroller 125 keeps tallies of the pills in the container and the pills that have been dispensed and the pills not dispensed, a Tilt Sensor 120 knows the relative position of the Reporting Pill Dispenser, when the Reporting Pill Dispenser is inverted (dumping, upside down), the Microcontroller 125 enables a signal to the Pill Dispensing Mechanism 108 to dispense a pill, a Real Time Clock 140 to know the time of day and set alarms for pill dispensing events. A Battery 121 of adequate size to energize the Reporting Pill Dispenser for the duration of the prescription period. The Reporting Pill Dispenser contains a Pill Funnel 113 to align the pills in a sequential order before the Pill Dispenser Mechanism 108.

The Reporting Pill Dispenser enhances user compliance for following through a particular drug prescription period (regimen, duration) by reminding the user when pills are to be taken, offsetting negative effects of memory loss and other cognitive dysfunctions, attention span disorder, attenuation of special senses, lack of user education, and knowing when the user attempts over dosage, etc. The Reporting Pill Dispenser reminds users and dispenses pills only to authorized users at appropriate times.

When the Reporting Pill Dispenser is first issued into service the Physician, Pharmacy, or Hospital 135, or User 128 fills the container with the prescribed pills. The Reporting Pill Dispenser can accommodate common sizes of tablets and capsules sized pills. The Physician, Pharmacy, or Hospital 135, or User 128 enters the prescription into the Reporting Pill Dispenser by entering data into the User Database 136, then snaps the cap onto the container, not able to be removed. The process of entering data into the User Database 136, enters the pill dosage number of pills, time of day intervals, total pill count, and user authentication of the Reporting Pill Dispenser. The User's fingerprint is down loaded into the Reporting Pill Dispenser for reference, this fingerprint is retained in the Reporting Pill Dispenser until the disposal of the Reporting Pill Dispenser.

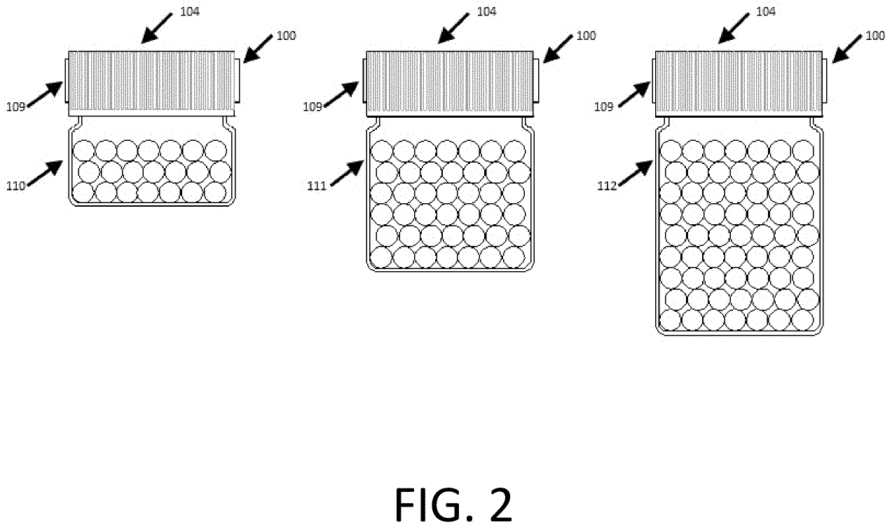

FIG. 2 illustrates the different Pill Container sizes that are envisioned in the embodiment, sizes of the Pill Containers are dependent on the quantity of pills prescribed for use in the Reporting Pill Dispenser. Three Pill Container sizes are shown, small size pill container for less than 30 pills (Pill Container 110), medium size pill container for less than 60 pills (Pill Container 111), and large size pill container for less than 90 pills (Pill Container 112) per prescription. Each Pill Container contains the Cap Retention Mechanism 114 which when applied prevents the Pill Dispenser Cap 104 from being removed.

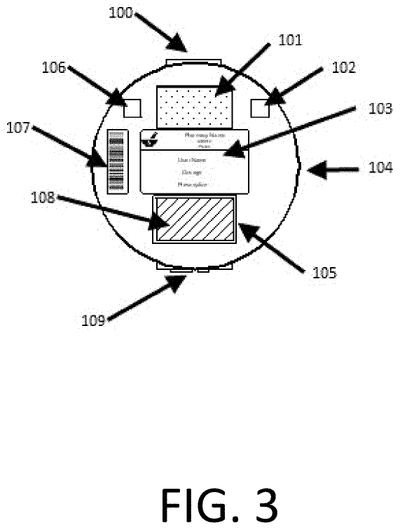

FIG. 3 illustrates the top view of the Reporting Pill Dispenser, showing the Pill Dispenser Cap 104, the Pharmacy Label 103 applied to the Pill Dispenser Cap 104, the YES Switch 102, for affirmative responses to questions on the Information Display 101, the No Switch 106, for negative responses to questions on the Information Display 101, the User Authentication Device 100 to authenticate the current user of the Reporting Pill Dispenser, the Serial Number 107 bar code label of the Reporting Pill Dispenser, each Serial Number 107 for the Reporting Pill Dispenser is unique, the Pill Dispensing Mechanism 108 which dispenses pills from the interior of the Pill Container 110 to the exterior of the Reporting Pill Dispenser and inputs pills from the exterior of the Reporting Pill Dispenser to the interior of the Pill Container 110 under control by the Microcontroller 125, the Pill Slot 105 which provides a passage from the interior of the Pill Container 110 to the exterior of the Reporting Pill Dispenser, the Bio-Sensor 109 which contains the Heart Rate Monitor 116 and the Skin Temperature Sensor 117 to measure the health of the User 128 by taking measurements on the thumb.

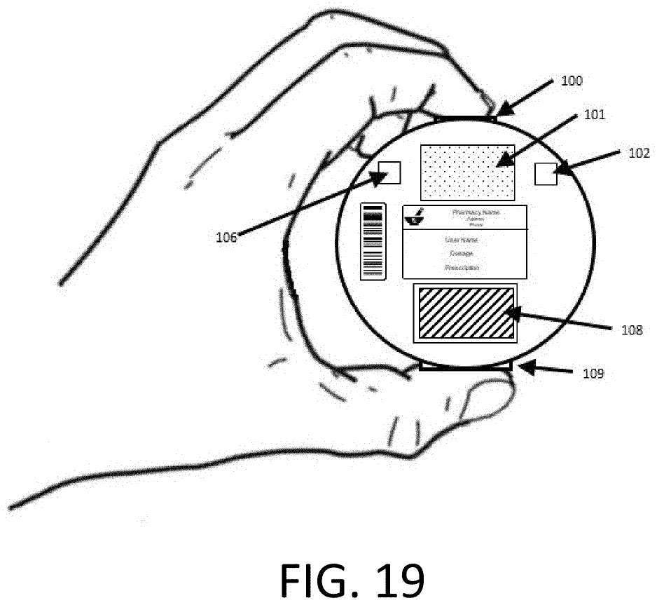

FIG. 19 illustrates the User 128 interaction with the Reporting Pill Dispenser. The User 128 holds the Reporting Pill Dispenser between the index and thumb fingers of the left or right hand whichever is convenient. The thumb is applied to the Bio-Sensor 109 where the Heart Rate Monitor 116 measures the heart rate and calculates the oxygen level in the blood is calculated, and the blood pressure reading is calculated. The thumb is also where the skin temperature is measured by the Skin Temperature Sensor 117. The Bio-Sensor 109 has a Bio-Sensor Touch Sensor 115 to know when the User 128 is touching the Reporting Pill Dispenser, and a Bio-Sensor Switch 129 to know when the User 128 applies pressure between the thumb and index finger (pinching action). The index finger (any finger) is applied to the User Authentication Device 100, which reads the fingerprint by the Fingerprint Reader 119 for that finger. Any finger can be used with the Reporting Pill Dispenser, as long as the User 128 is consistent. The User Authentication Device 100 has a User Authentication Touch Sensor 118 incorporated into the Fingerprint Reader 119 to know when the User 128 touches the User Authentication Device 100.

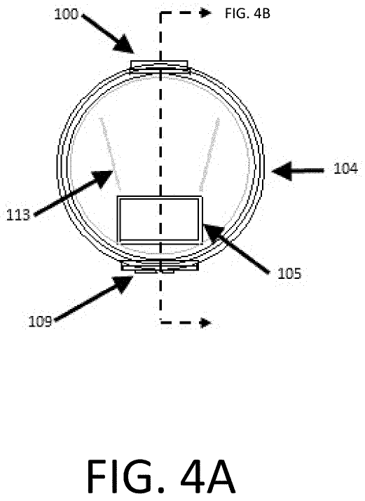

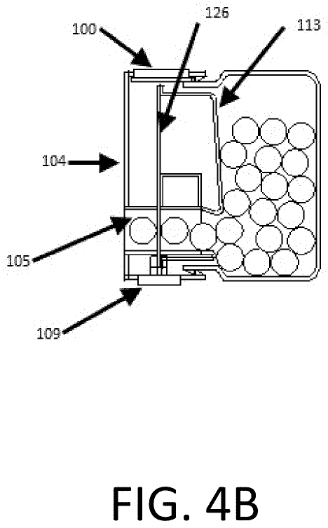

FIG. 4A illustrates the interior Pill Funnel 113 that is attached to the bottom side of the Microcontroller Assembly 126. The Pill Funnel 113 aligns the pills in an appropriate alignment so that they can enter the Pill Slot 105 without clogging. Pills enter the Pill Funnel 113 when the Reporting Pill Dispenser is turned upside down as in pouring from the reporting Pill Dispenser. The pills enter the Pill Slot 105 one at a time to be dispensed by the Pill Dispenser Mechanism 108. Several embodiment of the Pill Funnel 113 are envisioned, one for capsules and a separate for tablets. The appropriate Pill Funnel 113 is snapped into place by the Pharmacy depending on the medication being used in the Reporting Pill Dispenser.

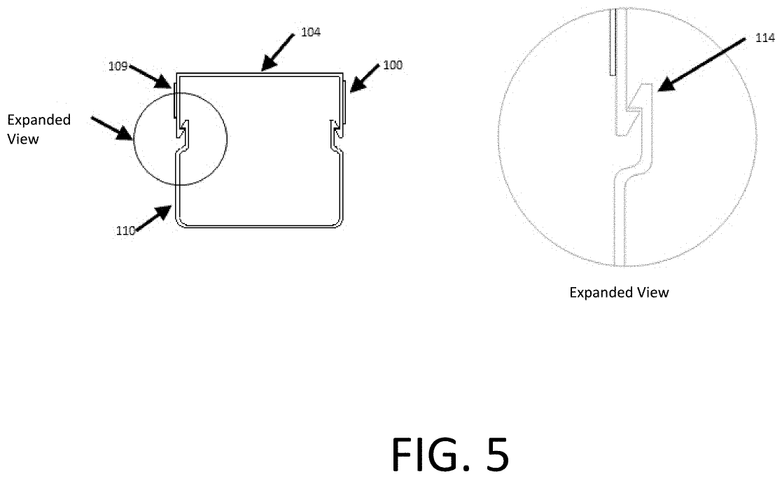

FIG. 5 illustrates the Cap Retention Mechanism 114, which allows the Pill Dispenser Cap 104 to be installed on the Pill Container 110, but not able to be removed. The Cap Retention Mechanism 114 is retained to the Pill Container 110, 111, 112 and cannot be removed without destroying the Reporting Pill Dispenser. This Cap Retainer Mechanism 114 is incorporated to prevent children from opening the Pill Container 110.

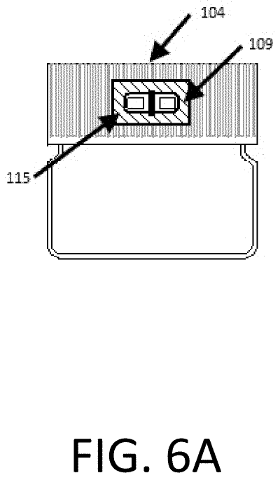

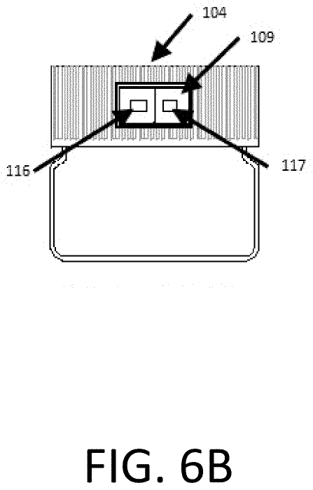

FIG. 6A and FIG. 6B illustrates a close up view of the Bio-Sensor 109, showing the Bio-Sensor Touch Sensor 115, with a window for the Heart Rate Monitor 116 and the Skin Temperature Sensor 117 to make contact with the User's 128 thumb. FIG. 6B shows the Heart Rate Monitor 116, and the Skin Temperature Sensor 117, without the Bio-Sensor Touch Sensor 115 shown.

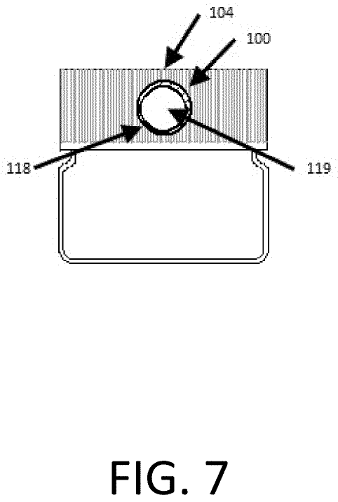

FIG. 7 illustrates a close up view of the User Authentication Device 100, showing the User Authentication Touch Sensor 118 (ring around the Fingerprint Reader 119), and the Fingerprint Reader 119.

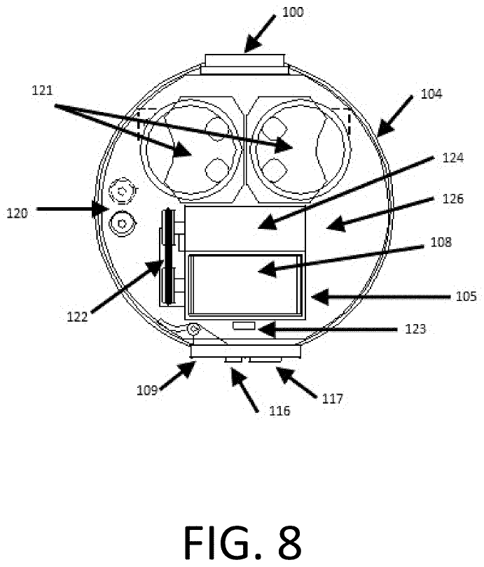

FIG. 8 illustrates a bottom view of the Reporting Pill Dispenser, without the Pill Funnel 113 shown. The bottom side of the Microcontroller Assembly 126 consists of two Batteries 121 to power the Reporting Pill Dispenser for the duration of the prescription, a Tilt Sensor 120 to know the relative position of the Reporting Pill Dispenser (upright or upside down), the Pill Dispensing Mechanism 108 which dispenses a pill from the inside of the Pill Container 110 to the exterior of the Reporting Pill Dispenser by the Pill Slot 105, the Motor 124 and the Pulley 122 of the Pill Dispenser Mechanism 108 which spins the Pill Tray 137 under control by the Microcontroller 125. Pills are detected in the Pill Slot 105 by the Pill Slot Sensor 123, thereby knowing if pills are in line to be dispensed, and the Pill Slot Sensor 123 can detect if pills pass through the Pill Slot 105.

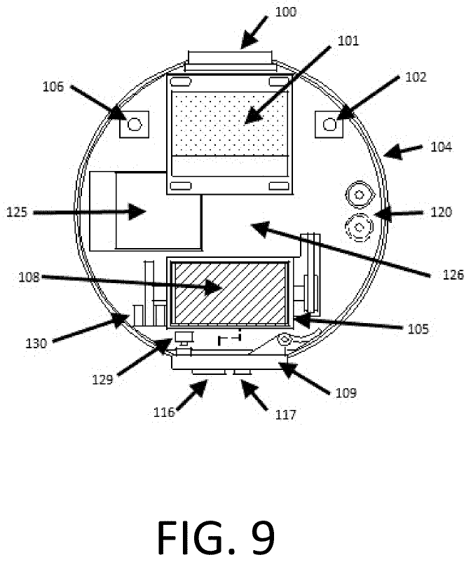

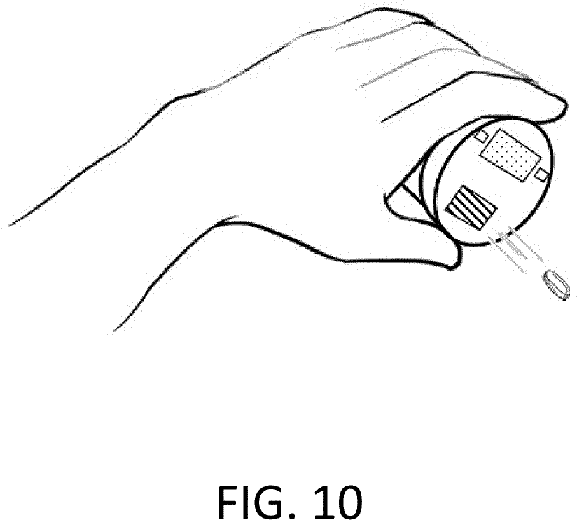

FIG. 9 illustrates a top view of the Reporting Pill Dispenser, without the Pill Dispenser Cap 104, revealing the Microcontroller Assembly 126 top view. The top view of the Microcontroller Assembly 126 consists of the Microcontroller 125, the User Authentication Device 100 connected to the Microcontroller Assembly 126, the Information Display 101, the YES Switch 102 for affirmative responses to questions on the Information Display 101, the No Switch 106 for negative responses to questions on the Information Display 101, the User Authentication Device 100 to authenticate the current user of the Reporting Pill Dispenser, the Pill Dispensing Mechanism 108 which dispenses pills from the interior of the Pill Container 110 to the exterior of the Reporting Pill Dispenser and dispenses pills from the exterior of the Reporting Pill Dispenser to the interior of the Pill Container 110 under control by the Microcontroller 125, the Pill Slot 105 which provides a passage from the interior of the Pill Container 110 to the exterior of the Reporting Pill Dispenser, the Bio-Sensor 109 which contains the Heart Rate Sensor 116 and the Skin Temperature Sensor 117 to measure the health of the User 128 by taking measurements on the thumb of the User 128, the Tilt Sensor 120 to measure the relative position of the Reporting Pill Dispenser, the Position Sensor 130 of the Pill Dispensing Mechanism 108 to indicate the position of the Pill Tray 137 (face up or face down), and the Bio-Sensor Switch 129 to signal when the User 128 presses the Bio-Sensor 109 indicating the User's 128 interaction with the Reporting Pill Dispenser.

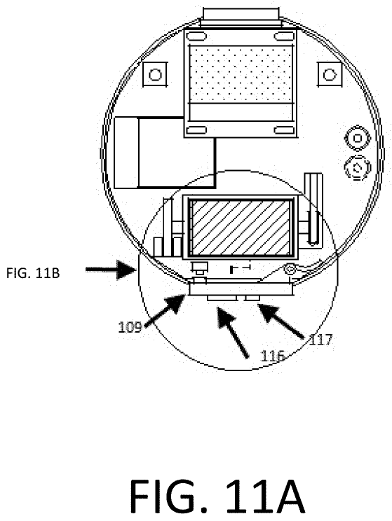

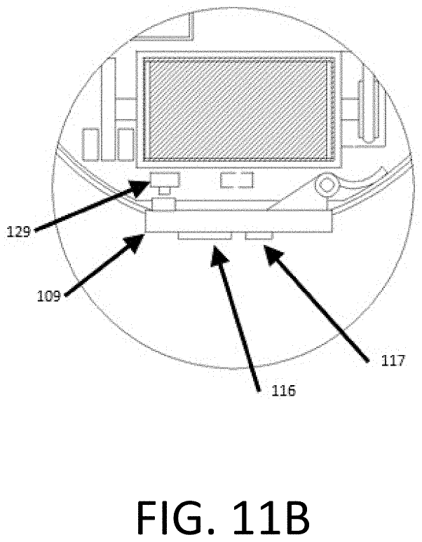

FIG. 11 illustrates a close up view of the Bio-Sensor Switch 129 which signals when the User 128 presses the Bio-Sensor 109 indicating the Users 128 interaction with the Reporting Pill Dispenser.

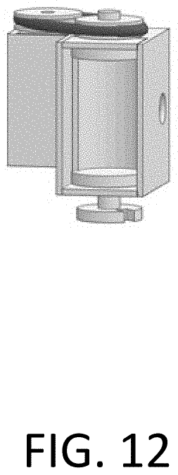

FIG. 12 shows an isometric view of the Pill Dispenser Mechanism 108.

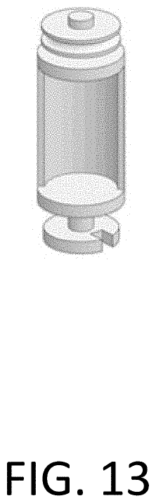

FIG. 13 shows an isometric view of the Pill Tray 137 of the Pill Dispenser Mechanism 108.

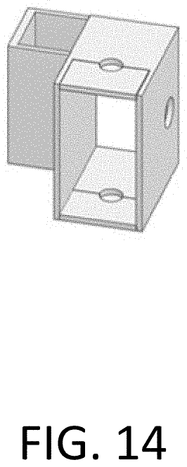

FIG. 14 shows an isometric view of the Pill Slot 105 of the Pill Dispenser Mechanism 108.

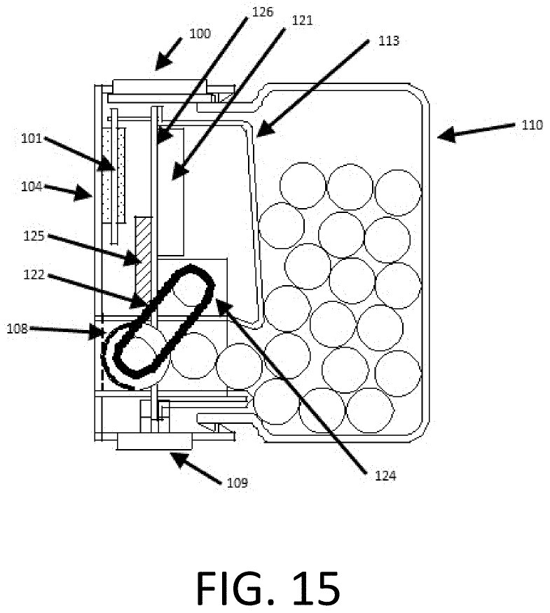

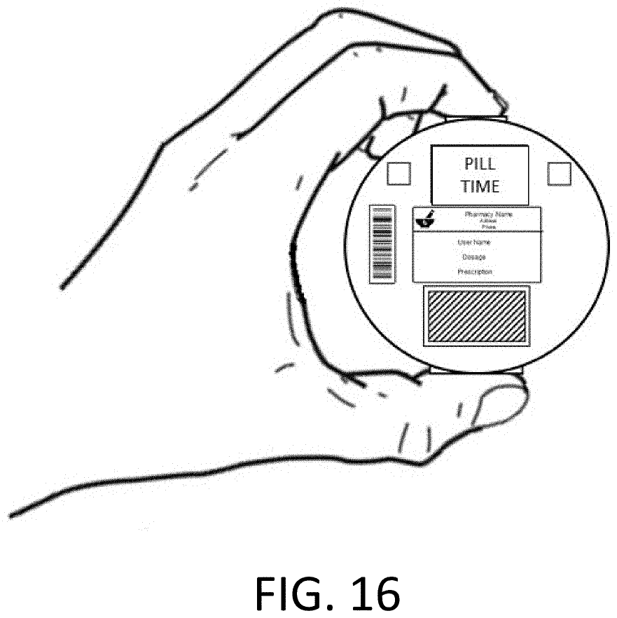

FIG. 15 shows a detailed view of the Pill Dispenser Mechanism 108 drive Motor 124 and Drive Pulley 122. In this side view the Reporting Pill Dispenser is horizontal with the pills in the Pill Container 110 falling in line with the Pill Funnel 113. As the Reporting Pill Dispenser continues to proceed upside down (counterclockwise) the pills continue to align with the Pill Slot 105, the Tilt Sensor 120 detects the Reporting Pill Dispenser inverted nature (dumping, upside down), enabling the Pill Dispensing Mechanism 108 and Motor 124 by control of the Microcontroller 125.

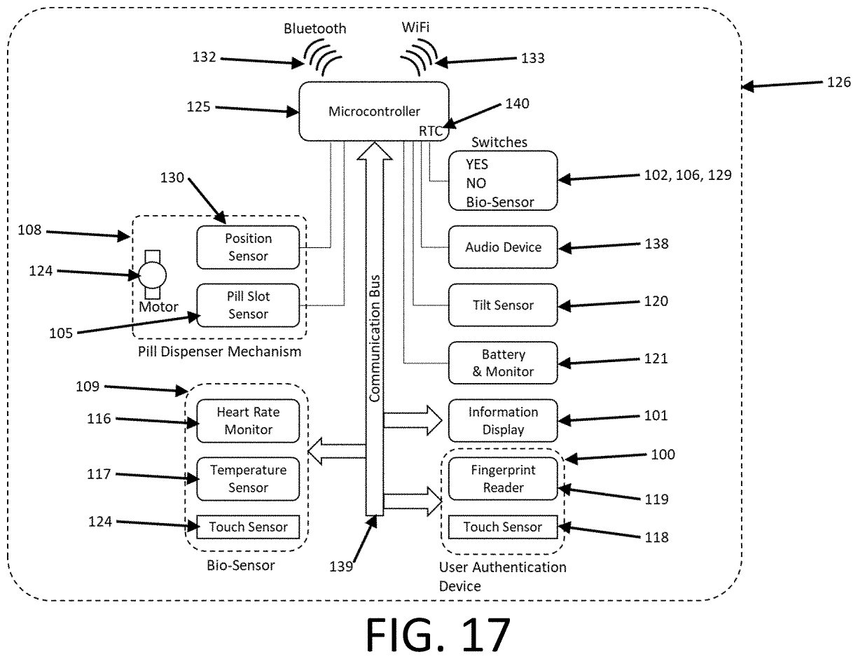

FIG. 17 illustrates in block diagram form one embodiment of the present system for the Microcontroller Assembly 126, the Microcontroller 125 is the heart of the Reporting Pill Dispenser. The Microcontroller 125 controls all other components in the Microcontroller Assembly 126. The Microcontroller Assembly 126 consists of the Pill Dispenser Mechanism 108, the Bio-Sensor 109, User Authentication Device 100, plurality of switches 102, 106, 129, an Audio Device 138, a Tilt Sensor 120 to know when the Reporting Pill Dispenser is inverted (dumping, upside down), a Battery 121 capable of powering the Reporting Pill Dispenser for the duration of the prescription, and an Information Display 101 where queries and interaction with the User 128 is performed. The Microcontroller 125 communicates with the Bio-Sensor 109, User Authentication Device 100, and Information Display 101 by a Communications Bus 139. The other elements of the Microcontroller Assembly 126 are controlled by logical pins on the Microcontroller 125.

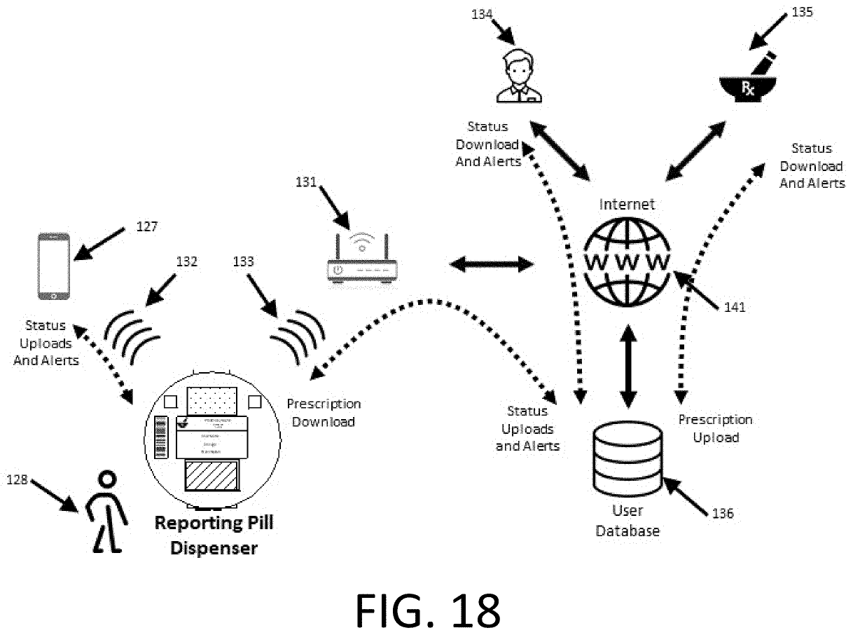

FIG. 18 illustrates the communication systems outside the Reporting Pill Dispenser, consisting of the Users 128 or Family members 134 associated with the User 128, a Smartphone 127 that has ability to communicate with the Reporting Pill Dispenser and provide configuration setup, a Home Router 131 with WiFi wireless interface 133 connectivity which the Reporting Pill Dispenser will communicate with the User Database 136, the Family members 134 who have privileges to receive the status information from the User Database 136, the Pharmacy, Physician, Pharmacy, or Hospital 135 who prescribed the medication for the User 128, the User Database 136 which retains the data from the Reporting Pill Dispenser system 136 and provides interfaces for the Family members 134 and the Physician, Pharmacy, or Hospitals 135 associated with the User 128, the Bluetooth wireless interface 132 between the Reporting Pill Dispenser and the Smartphone 127 providing configuration setup, the WiFi wireless interface 133 between the Reporting Pill Dispenser and the Home Router 131, the Internet 141 network which provided communication connectivity between the Reporting Pill Dispenser, the User Database 136, the Family members 134 and Physician, Pharmacy, or Hospitals 135 that require access to the user data in the User Database 136.

FIG. 19 illustrates the user holding the Reporting Pill Dispenser in his right hand. The Reporting Pill Dispenser can be handled in either the right hand or the left hand whichever is convenient. The Reporting Pill Dispenser is used with the Information Display 101 at the top, with the Yes Switch 102 and No Switch 106 accessible by the other hand. The user applies the thumb on the Bio-Sensor 109 and the index finger on the User Authentication Device 100, by applying pressure the Bio-Sensor Switch 129 is engaged to indicate the User 128 is engaged with the Reporting Pill Dispenser.

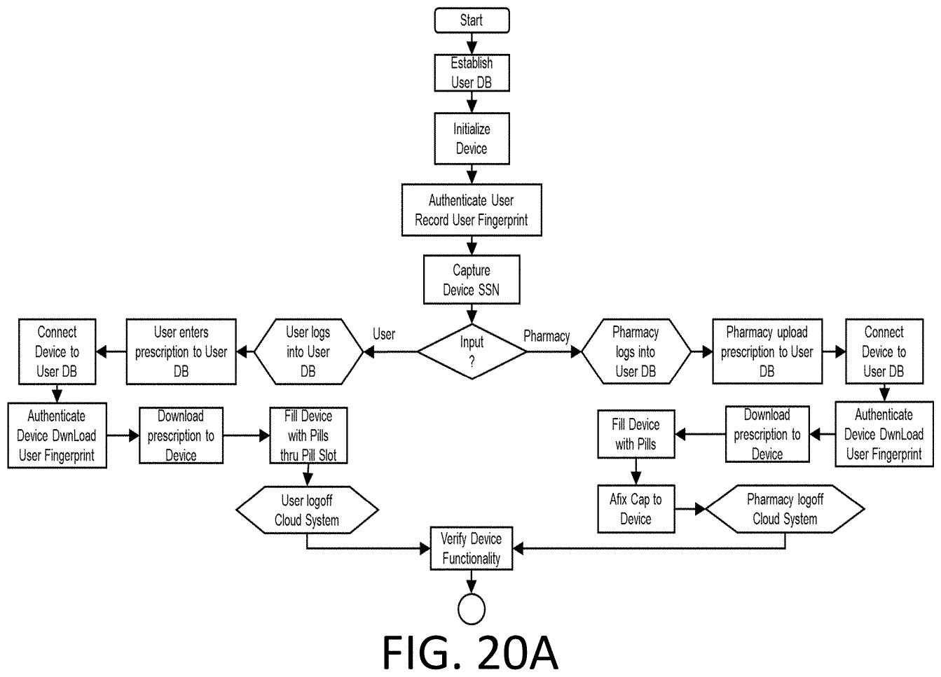

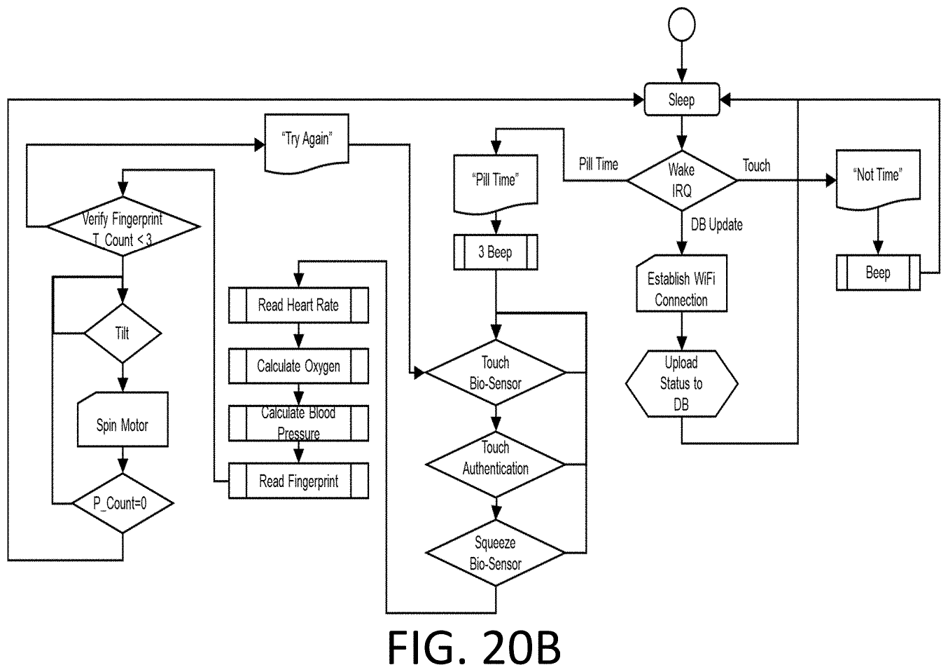

FIG. 20A and FIG. 20B show the general flow for the method of management for the Reporting Pill Dispenser. This is a high level program flow for the interaction of the user with the Reporting Pill Dispenser. In general the Reporting Pill Dispenser is commissioned into service at the Pharmacy that dispenses the prescription for the User 128. The Reporting Pill Dispenser is to be managed from the Pharmacy like any other equipment administered for medication reasons. The flow begins with the User 128 establishing an account and login credentials for a User Database 136, then the User 128 logs into the User Database 136 to record the User's 128 fingerprint, next the User Database 136 is connected to the Reporting Pill Dispenser to capture the device Serial Number 107, each Reporting Pill Dispenser has a unique serial number. A decision is made next, if the prescription information is to be entered by the Pharmacy or the user.

If the prescription is entered by the Pharmacy, the Pharmacy first logs into the User Database 136, this login establishes the credentials between the Pharmacy, the User 128, and the Reporting Pill Dispenser for the User's 128 prescription. The user can have one or more Reporting Pill Dispensers with the Pharmacy with more than one Pharmacy. The Pharmacy next uploads the User's 128 prescription for the medication prescribed. This prescription information contains as a minimum the drug prescribed, the usage information (when taken, times, amounts to be taken, etc.), the beginning pill count, the refill dynamics, and the recorded fingerprints from when the User 128 established an account with the User Database 136. Next the User Database 136 is connected to the Reporting Pill Dispenser, the information related to this user for this prescription is downloaded into the Reporting Pill Dispenser once the User 128 authenticates with the Reporting Pill Dispenser at the Pharmacy. Next the Pharmacy would fill the Reporting Pill Dispenser with the appropriate pill count and affix the Pill Dispenser Cap 104 on the Pill Container 110.

If the prescription is entered by the User 128, the user first logs into the User Database 136, this login establishes the credentials of the user, and the Reporting Pill Dispenser for the User's 128 medication. The user can have one or more Reporting Pill Dispensers at any one time. The user next uploads the User's 128 prescription for the medication. This upload process is managed by the User 128 entering information into a Smartphone 127 which is logged into the Reporting Pill Dispenser device. This prescription information contains as a minimum the drug prescribed, the usage information (when taken, times, amounts to be taken, etc.), the beginning pill count, the refill dynamics, and the recorded fingerprints from when the User 128 established an account with the User Database 136. Next the User Database 136 is connected to the Reporting Pill Dispenser, the information related to this User 128 for this prescription and downloaded into the Reporting Pill Dispenser once the User 128 authenticates with the Reporting Pill Dispenser.

Next the User 128 will feed the pills one at a time to the Pill Slot 105, push the Yes Switch 102 in the Reporting Pill Dispenser to signify the pill is ready to be passed into the container. The Microcontroller 125 spins the Pill Tray 137 passing the pill from the exterior of the Reporting Pill Dispenser to the interior of the Pill Container 110. As the pill passes through the Pill Slot 105 the Pill Slot Sensor 123 counts the pills entered into the Pill Container 110.

After the pills have been entered into the Pill Container 110, the User 128 logs off the User Database 136 and the Reporting Pill Dispenser goes into sleep mode.

The Reporting Pill Dispenser awakens from sleep mode by one of three possible indications, pill time event alarm (pill time), User 128 touches the Bio-Sensor 109 or User Authentication Device 100, or the required periodically update of the User Database 136.

The pill time event is indicated by the Real Time Clock 140 alarm being activated, the real time alarm is programmed for the times when the prescription needs to be taken. The real time alarm is auto reset after the event until the alarm is canceled. Next the Reporting Pill Dispenser displays “Pill Time” on the Information Display 101 followed by an audial alarm on the Audible Alert Device 138 signifying to the user it's time to take a pill. The User 128 touches the Bio-Sensor 109 followed by touching the User Authentication Device 100, then pressing the Bio-Sensor 109 until the Bio-Sensor Switch 129 engages. The Informational Display 101 tells the user to “dump” the pill out the Pill Slot 105. The user inverts the Reporting Pill Dispenser until the Tilt Sensor 120 signifies the Pill Container 110 is inverted (dumping, upside down) by the Tilt Sensor 120. Once inverted (dumping, upside down) the Microcontroller 125 engages the Motor 124 of the Pill Dispensing Mechanism 108 to deliver a pill from the interior of the Pill Container 110 to the exterior of the Reporting Pill Dispenser. If this is a single pill dosage event the Reporting Pill Dispenser signifies the user the pill dispensing event is complete, and the Reporting Pill Dispenser goes back to sleep. If this pill dispense event requires another pill to be dispensed the Reporting Pill Dispenser informs the user to return the Reporting Pill Dispenser upright as indicated by the Tilt Sensor 120, then begin another pill dispensing event as before. This process is continued until all pills are dispenser during this pill dispensing event. Again once the pill dispensing event is complete the Reporting Pill Dispenser goes back to sleep waiting on the next pill event.

The User Database 136 upload event is indicated by the Real Time Clock 140 alarm being activated, the real time alarm is programmed for the times when the User Database 136 upload is to be executed. This User Database 136 upload can occur after every pill dispensing event, or hourly, or daily, or weekly, etc., as required. The real time alarm is auto reset after the event until the alarm is canceled.

The user touch event is indicated by the User 128 touching the Bio-Sensor 109 or the User Authentication Device 100. Once the User 128 touches the Reporting Pill Dispenser the Information Display 101 and operatively connected Microcontroller 125 would go through an interaction with the User 128 to identify what the reason for touching the Reporting Pill Dispenser, and processing of the touch until conclusion.

1. A Reporting Pill Dispenser device for dispensing pills, capsules, or tablets, “pills”, to users in a convenient, secure, and portable design; following a pre-prescribed prescription regimen for pill dosage, including a Bio-Sensor for user health monitoring, a User Authentication Device to verify the User, dosage tracking and dosage reporting to a User Database. The device comprises: a) a Pill Dispenser Container Cap; b) a Pill Dispenser Container sized to hold a plurality of pills prescribed; c) a Pill Slot within the Pill Dispenser Container Cap of appropriate size to allow pills to pass one at a time therethrough, the Pill Slot allows the transfers of pills from the Pill Dispenser Container interior to the exterior of the Pill Dispenser Container Cap, and from the exterior of the Pill Dispenser Container Cap to the Pill Dispenser Container; d) a Pill Dispenser Mechanism capable of dispensing one pill at a time or one dosage at a time; e) a Pill Funnel to align the pills for dispensary into the Pill Dispenser Mechanism one at a time; f) a Microcontroller Assembly containing the electronic circuitry to dispense pills one at a time for an authorized user, and to communicate with a User Database regarding the pills dispensed, and to communicate with a Smartphone about the status of the Reporting Pill Dispenser.

2. The Reporting Pill Dispenser of claim 1 further comprising: a Pill Funnel to align the pills for dispensary within the container, the Pill Funnel aligns the pills by stacking the pills in a vertical column through a serpentine channel, aligning the capsules with adequate rotation to enter the Pill Dispenser Mechanism without clogging.

3. The Reporting Pill Dispenser of claim 1 further comprising: an Information Display visible through the Pill Dispenser Cap, the Information Display is the primary interface between the user and the Reporting Pill Dispenser.

4. The Reporting Pill Dispenser of claim 1 further comprising: a plurality of switches within the Pill Dispenser Cap (Yes Switch, No Switch, Bio-Sensor Switch), the switches are used for the user to respond to queries of the Information Display.

5. The Reporting Pill Dispenser of claim 1 further comprising: a User Authentication Device within the Pill Dispenser Cap, the User Authentication Device authenticates the user for pills dispensed to the user.

6. The User Authentication Device of claim 5 further comprising of a Fingerprint Reader, user authentication is performed by comparing current fingerprint readings against pre-recorded fingerprints of the user.

7. The Fingerprint Reader of claim 5 further comprising a User Authorization Touch Sensor to monitor the user touching the Fingerprint Reader.

8. The Reporting Pill Dispenser of claim 1 further comprising a: Bio-Sensor to monitor the user health, affixed to the Bio-Sensor lever of the Reporting Pill Dispenser, the Bio-Sensor lever engages the Bio-Sensor Switch of the Microcontroller Assembly of item 4 when pressure is applied to the Bio-Sensor.

9. The Bio-Sensor of claim 8 further comprising: a Heart Rate Monitor to measure the blood vessel blood flow in the Users thumb.

10. The Bio-Sensor of claim 8 further comprising: an Oxygen Monitor to calculate the blood oxygen levels based on data from the Heart Rate Monitor of claim 9 for the user.

11. The Bio-Sensor of claim 8 further comprising a Blood Pressure Monitor to calculate the blood pressure levels based on data from the Heart Rate Monitor of claim 9 for the user.

12. The Bio-Sensor of claim 8 further comprising: a Skin Temperature Monitor to measuring the skin temperature of the Users' thumb or finger.

13. The Bio-Sensor of claim 8 further comprising: a Bio-Sensor Touch Sensor to monitor the user touching the Bio-Sensor.

14. The Reporting Pill Dispenser of claim 1 further comprising: a Cap Retention Mechanism design to prevent the Pill Dispenser Cap removal once applied.

15. The Reporting Pill Dispenser of claim 1 further comprising: a Pill Dispenser Mechanism for the dispensing of pills one at a time, further comprising of a cylindrical rotating Pill Tray of adequate dimension to hold one pill at a time, a semicircular tray the length of the cylindrical rotating tray, allowing the cylindrical rotating tray to spin on its horizontal axes, contains a Positioning Sensor to indicate the home position of the rotatory tray, containing a Pulley affixed to the cylindrical rotating tray to spin the rotatory tray by the pill dispenser Motor activated by the Microcontroller, a Pill Slot Sensor for the detection of pills in the Pill Funnel.

16. The Reporting Pill Dispenser of claim 1 further comprising: a Microcontroller Assembly containing the electronic circuitry and components to control the Reporting Pill Dispenser; a Microcontroller, further comprising of a WiFi wireless interface for communications to a User Database, further comprising of a Bluetooth wireless interface for communications to a Smartphone devices in near proximity to the pill dispenser, and further comprising of a Real Time Clock to set alarms when the pill prescribed dosage must be administered; the Microcontroller is operatively connected to the Bio-Sensor of claim 8; the Microcontroller is operatively connected to the User Authentication Device of claim 5, the Microcontroller is operatively connected to the Pill Dispenser Mechanism of claim 1 and all its sub-claims, further comprising of the Pill Dispenser Mechanism of claim 15; the Microcontroller is operatively connected to the Information Display of claim 3; further comprising a Tilt Sensor to know the relative position of the pill dispenser; further comprising a Battery to energize the Reporting Pill Dispenser; and further comprising of an Audible Alert Device to alert the user.

17. A method for managing the pill dispensing regimen for the Reporting Pill Dispenser, the method comprising: storing a plurality of pills pre-prescribed in the Pill Container by the Physician, Pharmacy, or Hospitals; the pills are loaded into the Reporting Pill Dispenser by filling the Pill Container before affixing the non-removable Pill Dispenser Cap, or by inserting pills by way of the Pill Slot of the Pill Dispenser Cap by using an external funnel; the pill regimen is entered into the Reporting Pill Dispenser by way of the User Database by way of the WiFi wireless interface under program control, identifying events (times) and dosage of when the pills are to be administered; manually entering the times and dosage of when the pills are to be administered by the user; an Information Display visible on the exterior of the Reporting Pill Dispenser for the user to interact with the Reporting Pill Dispenser; and a Pill Dispenser Mechanism to dispense pills under control by the Microcontroller.

18. The method of claim 17 further comprising: a Microcontroller operatively connected to the Information Display; operatively connected to a User Authentication Device of claim 5; operatively connected to the Bio-Sensor of claim 8 for the User's health; operatively connected to the Pill Dispenser Mechanism of claim 15, to dispense one pill at a time or one dosage at a time, a pill dispenser Motor operationally controlled by the Microcontroller and configured to dispense pills from the Pill Container to the exterior of the pill dispenser, wherein the Microcontroller is configured to activate the pill dispenser Motor when the Real Time Clock generates an pill dispensing event, and the Reporting Pill Dispenser is inverted (dumping, upside down) from normal position as indicated by the Tilt Sensor.

19. The method of claim 18 further comprising: a Bio-Sensor to monitor the health of the user, user health is determined by measuring the heart rate of the user, calculating the oxygen rate of the user, calculating the blood pressure of the user, and measuring the skin temperature of the user.

20. The method of claim 18 further comprising: a User Authentication Device of claim 5 to authenticate the user by sampling current fingerprints of the user and compare said fingerprint to previously read fingerprints of the user.

21. The method of claim 17 further comprising: a communication method for the Microcontroller to interface by way of the WiFi wireless interface to the Internet of item 16 to the User Database which collects: User pill regimen execution data as prescribed by the Physician, Pharmacy, or Hospitals, User's regimen data of the pill regimen, User's health measurements data, User's authentication monitoring data, User's pill dispensing attempts data for non-regimen pill times, initial pill counts in the Pill Container, current pill counts in the Pill Container, count and time of pills dispensed from the Reporting Pill Dispenser, times of user interaction with the Reporting Pill Dispenser, and status of the Reporting Pill Dispenser.

22. The method of claim 17 further comprising: a communication method for the Microcontroller to interface by way of the Bluetooth wireless interface of item 16 to Smartphone devices in near proximity to the Reporting Pill Dispenser which collects: user pill regimen as prescribed by the Physician, Pharmacy, or Hospitals, user regimen data of the pill regimen, User's general health measurements data, User's authentication monitoring data, User's pill dispensing attempts data for non-regimen pill times, initial pill counts in the Pill Container, current pill counts in the Pill Container, count and time of pills dispensed from the Pill Container data, times of User's interaction with the Pill Container data, and status of the Reporting Pill Dispenser data.

23. The method of claim 17 further comprising: a method for the configuring of the Reporting Pill Dispenser to automatically administer pills on a pill regimen as defined by the Real Time Clock, as prescribed by the User's Physician, Pharmacy, or Hospitals.

24. The method of claim 17 further comprising: a method for the configuring of the Reporting Pill Dispenser to manually administer pills on a pill regimen as defined by the Real Time Clock, in a manual manner by the user.

25. The method of claim 17 further comprising: a method for the communication by way of the WiFi wireless interface of item 16 to a User Database for the storing of the data from the Reporting Pill Dispenser, this data contains the pill regimen sequences entered into the reporting pill dispenser, the times administered and dosage amounts when the pill are to be dispensed, the total count of the pills when filled in the reporting pill dispenser, the current count of the remaining pills in the reporting pill dispenser, and the recorded events as recorded by the Microcontroller when the user interacts with the Reporting Pill Dispenser.

26. The method of claim 17 further comprising: a method for the administrating of the pills in the Reporting Pill Dispenser, one at a time or one dosage at a time by spinning the Pill Dispensing Mechanism, cylindrical rotating Pill Tray when; the Reporting Pill Dispenser is signaled by the pill regimen Real Time Clock, time to dispense one pill or one dosage to the user and, the Reporting Pill Dispenser detects the user has an index finger or thumb on the User Authentication Device and, the user is authenticated by the Fingerprint Reader and, the user has a thumb or index finger on the Bio-Sensor and, the user applies pressure to the Bio-Sensor Switch to engage the switch on the Bio-Sensor lever and, the user holds the Reporting Pill Dispenser in an inverted (dumping, upside down) position as indicated by the Tilt Sensor, the Pill Dispensing Mechanism is activated by the Microcontroller which spins the cylindrical rotating Pill Tray to delivering one pill to the exterior of the Reporting Pill Dispenser, additional pills are delivered to the exterior by continuing to spin the cylindrical rotating Pill Tray, pills that are delivered to the exterior are counted by the Pill Slot Sensor providing accurate counting of the pills dispersed, once all pills are dispensed per the dosage, the Microcontroller reverts to the beginning state, then reports the pill dispensing activity to the User Database for further processing.
